(19) United States
(12) Patent Application Publication
(10) Pub. No.: US 2023/0005071 A1
(43) Pub. Date: Jan. 5, 2023

(54) CANCER PATIENT GUIDANCE SYSTEM AND METHOD

(71) Applicant: HealthOme, Inc., Miami, FL (US)

(72) Inventors: Thomas R. Wamberg, Miami, FL (US); Warren T. Wamberg, Miami, FL (US); Bradley A. Perkins, San Diego, CA (US); Susan H. Darby, Wilmington, NC (US); David J. Stertzer, Wilmington, NC (US); Martin A. Sabarsky, Encinitas, CA (US); Darren Rowe, Fallston, MD (US)

(73) Assignee: HealthOme, Inc., Miami, FL (US)

(21) Appl. No.: 17/856,201

(22) Filed: Jul. 1, 2022

Related U.S. Application Data
(60) Provisional application No. 63/217,540, filed on Jul. 1, 2021.

(51) Int. Cl. G06Q 40/08

(52) U.S. Cl. CPC G06Q 40/08; G16H 10/60

(57) ABSTRACT

A system and method of combining omics-based health management resources and tools for the prevention, management, recovery and protection components within a critical illness or life insurance or assurance product. The system and method are configured to construct an individualized life assurance product for managing a risk of cancer based on a user's medical and behavioral history and genomics and genetic testing based on their biological data. The individualized life assurance product provides users with a cancer prevention component, a cancer management component, a cancer recovery component, and a financial protection component accessible via the customer portal. The components allow the user to reduce risk or prevent disease, manage active disease, recover from disease, and provide financial protection in the event of disease or death.

### CROSS-REFERENCE TO RELATED APPLICATION

The present application claims priority to, and the benefit of, U.S. Provisional Application No. 63/217,540, which was filed on Jul. 1, 2021 and is incorporated herein by reference in its entirety.

### FIELD OF THE INVENTION

The present invention generally relates to a system and method of creating and administering a life insurance or assurance product. More specifically, the present invention relates to a system and method of combining omics-based health management resources and tools for the prevention, management, recovery and protection components within a critical illness or life insurance or assurance product. Accordingly, the present specification makes specific reference thereto. However, it is to be appreciated that aspects of the present invention are also equally amenable to other like applications, devices and methods of manufacture.

### BACKGROUND

Life insurance or life assurance are contract based financial products between an insurance policy holder and an insurer. A traditional life insurance or critical illness policy typically provides cash at either death or in the event of the onset of a critical illness. The insurer guarantees a sum of money upon the death of the insured person that is payable to a designated beneficiary. Events such as terminal critical illnesses can also initiate payment depending on the specifics of the policy. The policy holder typically pays the insurer a lump sum or regularly occurring premium. The insurance benefits often include other expenses, such as medical and funeral expenses.

Life insurance or life assurance products are similar to other types of asset management products or systems. Asset management is a systematic approach to the governance and realization of value from the things over an entire life cycle that a group or entity is responsible for protecting. It may apply both to tangible assets and to intangible assets. Asset management is a systematic process of developing, operating, maintaining, upgrading, and disposing of assets in the most cost-effective manner.

Life-based insurance products tend to fall into two major categories: protection policies and investment policies. A protection policy is designed to provide a benefit, typically a lump-sum payment, in the event of a specified occurrence such as death. This is a type of term insurance. Investment policies are designed to facilitate the growth of collected capital by regular or single premiums. Common forms of investment policies include whole life, universal life, and variable life policies.

Insurance based products are typically limited to cash payouts with nothing more. There may be incentives built-in to the product to decrease premiums, but there are no support programs associated with the insurance products. Therefore, there is a need for a life insurance or assurance product that provides for prevention, management, recovery, and the financial impact of a catastrophic life event in the event of a significant illness, such as cancer.

In this manner, the improved system and method of the present invention accomplishes all of the forgoing objectives, thereby providing an easy solution of administering a life insurance or assurance product. A primary feature of the present invention a type of life insurance or assurance where an insurance company provides easier access to expensive and cutting edge technology which can help the policyholders live a longer and healthier life. Additionally, the method and system of the present invention democratize the cost of expensive technologies and make them affordable to the average person and creates shared-value, precision health management solutions.

The improved systems and methods create companion programs for the life, critical illness, and annuity insurance industry. The systems and methods are configured to create stand-alone “disease-specific” insurance policies or it can be embedded into existing insurance structures to help consumers proactively manage the risk of disease. The systems and methods are configured to help consumers navigate the clinical and financial challenges of managing and recover from diseases.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a system for creating and administering a life insurance or assurance product for managing a risk of cancer. The system comprises a non-transitory set of computer readable media hosted on a server comprising a memory storing a set of computer readable and executable software instructions configured as a customer portal. When executed by a processor, the server accesses a request from a processor of a user access device to create an individualized life assurance product. The server is further configured to upload a medical and behavioral history of a user to the customer portal.

Next, a plurality of genetic and genomic tests is suggested for the user based on an analysis of the user's medical and behavioral history. The system further comprises an at home cancer test kit comprising a plurality of genetic and genomic test materials. A user input of the test kit is used to access the server to guide the user through a genetic and genomic sampling procedure using the plurality of genetic and genomic test materials. Once the samples are collected, the plurality of genetic and genomic test materials are sent to a laboratory for analysis.

Once testing is complete, a plurality of test results are uploaded to the server for analysis by the set of computer readable software instructions and a user's cancer risk stratification based on the medical history, the behavioral history, and the plurality of genetic and genomic test results is generated. The set of computer readable software instructions then creates an individualized life assurance product for managing a risk of cancer based on the risk stratification analysis.

The individualized life assurance product comprises a cancer prevention component, a cancer management component, a cancer recovery component, and a financial protection component. The components are accessible via the customer portal. The individualized life assurance product is adjustable based on a plurality of the user's omics markers to regularly measure a health status of the user over the life of the individualized life assurance product or by utilizing a plurality of gamifying omics-based health management tools.

The subject matter disclosed and claimed herein, in another embodiment thereof, comprises a method for creating and administering a life assurance product. The method employs a computer-based system accessible via a user access device connected to the computer-based system over a network to accomplish the method. The method comprises constructing an individualized life assurance product based on a user's biological data. The use's biological data includes their genome and genetic profile.

The method begins by creating a customer portal. The customer portal is created on the computer-based system and is accessible via the user access device. A user then uses the user access device to request to create the individualized life assurance product. Next, the user access device is used to provide and upload a medical history of the user or to provide access to the user's electronic medical records to the customer portal. Similarly, the user access device is used to provide and upload a behavioral history of the user. The behavioral history may include a social or family history as well.

The method continues by recommending a plurality of genetic and genomic tests based on the user's biological data. A test kit is ordered and sent to the user to collect biological samples that are sent to a laboratory. Once the testing is completed, a plurality of genetic and genomic test results are received from the laboratory and uploaded to the customer portal.

Once the medical history, the behavioral history, and the test results are obtained, the computer-based system processes the data and generates a risk stratification individualized to the user based on the medical history, the behavioral history, and the test results. Next, the individualized life assurance product is curated based on the generated risk stratification. The individualized life assurance product may be adjusted over a life of the individualized life assurance product. Adjustment may be based on additional genetic and genomic test results, a plurality of omics-based health management tools, compliance with a customized health management strategy generated by the computer-based system, or by utilizing a plurality of gamifying omics-based health management tools.

The customer portal comprises a prevention component, a disease management component, a disease recovery component, and a risk based financial protection component. The prevention component is configured to recommend a genetic and genomic screening test plan, a genetic counseling plan, and an action plan based on the medical history, the behavioral history, and the plurality of genetic and genomic test results. The disease management component is configured to provide a medical advocate, an expert medical review of test results, a comprehensive genetic profile, a health record collation, and a care pathway review. The disease recovery component is configured to provide a continuing navigational support, a recurrence monitoring plan, and a personalized nutritional support. The risk based financial protection component provides the user a financial incentive based on the risk stratification for the user.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a block diagram of an exemplary computer system and a network environment of the present invention for implementing and administering a life assurance product in accordance with the disclosed architecture.

FIG. 2 illustrates a block diagram of a life assurance product accessible via a customer portal for use with the system and method of implementing and administering the life assurance product in accordance with the disclosed architecture.

FIG. 3 illustrates a flowchart of a method of implementing and administering the life assurance product in accordance with the disclosed architecture.

FIG. 4 illustrates a perspective view of a test kit for use with the system and method of implementing and administering a life assurance product in accordance with the disclosed architecture.

FIG. 5 illustrates a flowchart of an exemplary post-diagnostic user's journey using the system and method of implementing and administering a life assurance product in accordance with the disclosed architecture.

FIG. 6 illustrates a flowchart of creating and activating an account on a customer portal using the system and method of implementing and administering a life assurance product in accordance with the disclosed architecture.

FIG. 7 illustrates a flowchart of an exemplary physician review using the system and method of implementing and administering a life assurance product in accordance with the disclosed architecture.

FIG. 8 illustrates a flowchart of an exemplary test kit order process using the system and method of implementing and administering a life assurance product in accordance with the disclosed architecture.

FIG. 9A illustrates a flowchart of an exemplary physician review using the system and method of implementing and administering a life assurance product in accordance with the disclosed architecture.

FIG. 9B illustrates a flowchart continuing the exemplary physician review using the system and method of implementing and administering a life assurance product in accordance with the disclosed architecture.

FIG. 9C illustrates a flowchart continuing the exemplary physician review using the system and method of implementing and administering a life assurance product in accordance with the disclosed architecture.

FIG. 10 illustrates a flow chart of a data flow for a proactive screening using the system and method of implementing and administering a life assurance product in accordance with the disclosed architecture.

FIG. 11 illustrates a flow chart of a data flow for post diagnosis services using the system and method of implementing and administering a life assurance product in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, provides systems and methods for providing access to a range of genetic and genomic tests to help prevent and manage diseases, including cancers. The systems and methods are designed to constantly appraise and curate the life science landscape to ensure that policyholders have access to the most up to date and clinically actionable tests. The systems and methods allows for easier negotiated pricing and contracts with testing labs. A unique test sustainable material kit experience (box, buccal swabs etc.) with high regard for the environment is provided for testing and tailored to the individual.

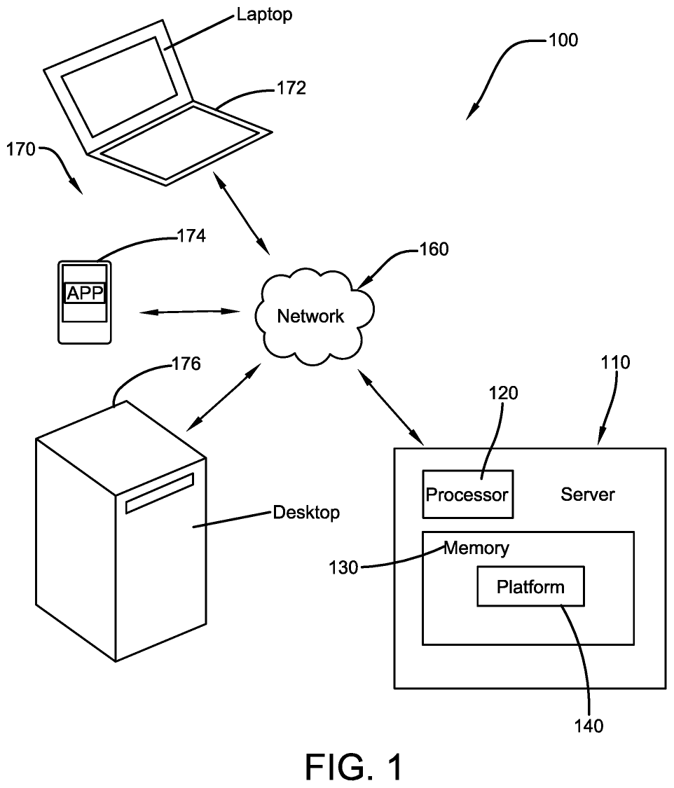

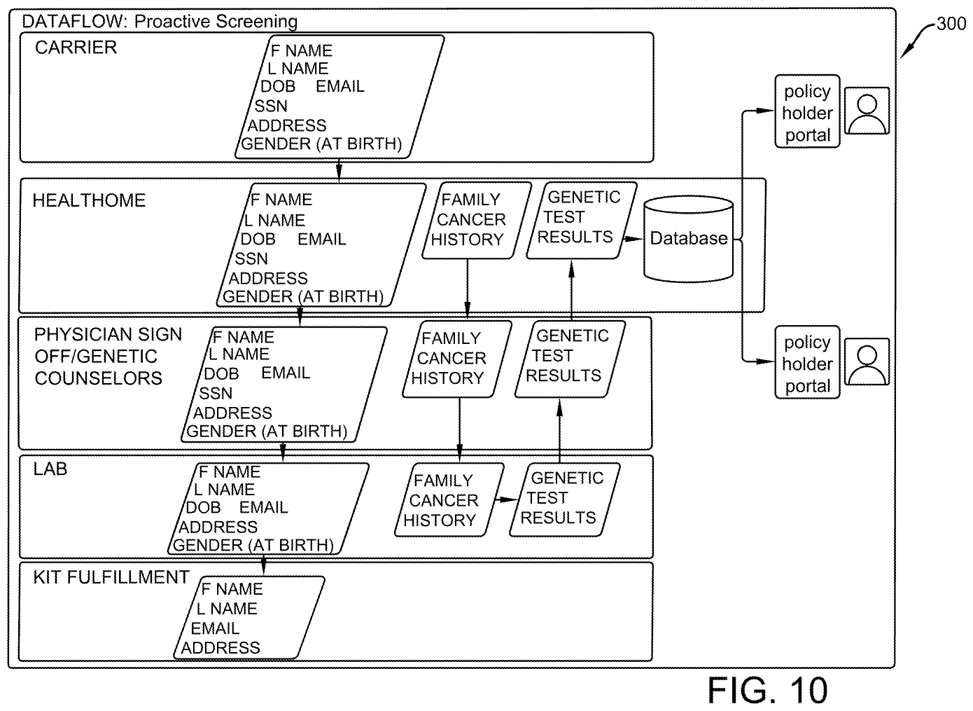

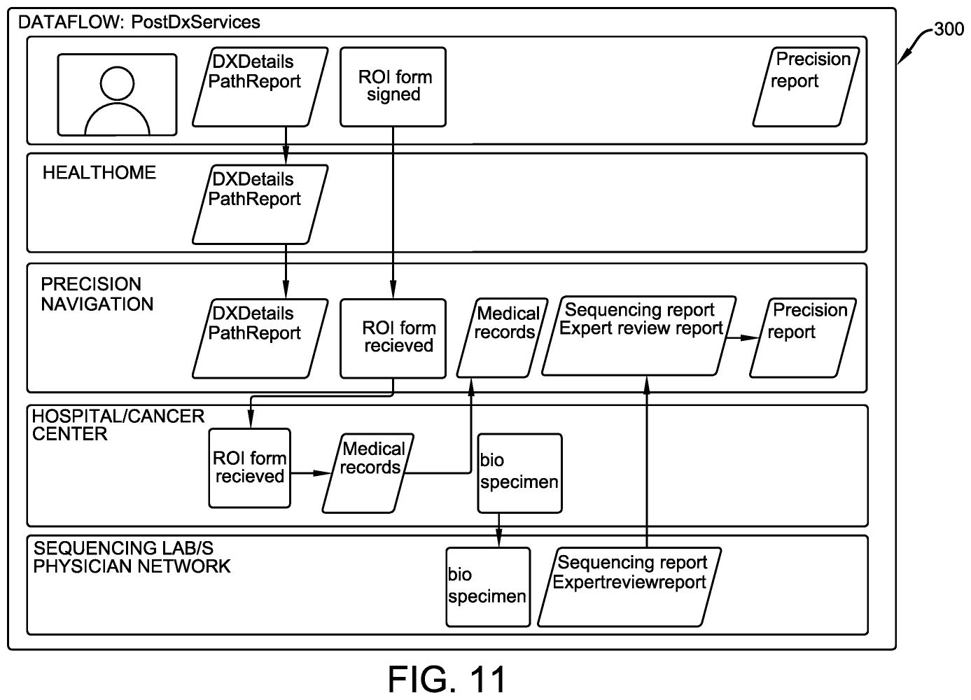

Referring initially to the drawings, FIGS. 1-11 illustrate systems and methods for creating and administering a life insurance or assurance product. The term “life assurance product” generally refers to a traditional insurance product or annuity insurance product combined with a life-optimization or whole health management plan configured with additional protections as described herein.

FIGS. 1-4 illustrate a system 100 for creating and administering a life assurance product for managing a risk of cancer. The life assurance product may be a critical illness policy, a life insurance policy, or a hybrid critical illness and life insurance policy. The life assurance product may be cancer-based, cardiovascular-based, cerebrovascular-based, or disease-specific, such as diabetes, genetic disorders, autoimmune diseases, a combination of the above, or the like.

The life assurance product of the present invention comprises a combination of prevention, management, recovery, and financial protections within a critical illness or life insurance product. There are no known existing products that combine all these elements into one product. Advantages of the system 100 include better mortality rates, better policyholder engagement, improved lapse ratios/better persistency, enhanced policyholder convenience, enhanced policyholder experience, improved longevity, better quality of life, dramatically reduced time to access innovative health management tools (bench to bedside), dramatically improve affordability to innovative omics based health management tools.

The life assurance product further combines omics-based health management resources and tools into a digitally accessible application where the user will be able to access their policy benefits and manage their health, with a life or critical illness insurance policy. The life assurance product life assurance product also includes gamifying omics-based health management to reduce premium amounts.

The life assurance product may be configured as a hybrid critical illness, and life policy similar to a life policy with an accelerated death benefit. Policyholders will be able to reduce their premium based on their biological age versus their chronological age. The system 100 includes a wide variety of omics based tests, educational resources, and support services accessible within one frictionless digital environment. The system may also have an option to include genetic testing for pets, fertility testing and prenatal testing for couples planning a family and annuity options creating a comprehensive life insurance or “assurance” policy.

The term “omics” refers to a pool of technologies used to measure and functionally characterize different biomolecules in cells or tissues. One of the primary purposes of omics is to study genomics. Genomics is the study of the whole genome, the complete set of DNA, of a person. The structural and functional characterization of the genome is performed using a combination of recombinant DNA, DNA sequencing and mapping methods, and bioinformatics. Functional genomics focuses on looking at the involvement of genes in shaping different biological activities. The main aim is to assess differential expressions of genes in a specific condition, such as during the development and progression of diseases, such as cancers.

Omics technologies have made it possible to study the genetic makeup of an individual leading to personalized medicines. Omics-based testing is useful for understanding the complex interactions between multiple genetic and environmental factors that occur during the pathogenesis of severe diseases, including cancers and cardiovascular diseases. Such information is particularly required to develop diagnostic and therapeutic approaches, as well as to study the clinical efficacy of specific drugs. The system 100 may be further configured to gamify “omics literacy” to encourage policyholders to become more aware about how their health is impacted and managed by omics technologies

Pharmacogenomics is a field of precision medicine designed to tailor specific medical treatment to each individual person or to a group of people. Pharmacogenomics tests and evaluates how a person's DNA affects the way that individual responds to drugs. #Drugs interact with each unique body in numerous ways, depending both on how the drug is taken and where in the body the drug acts as the body needs to break a drug down and get it to the intended area.

Examples of these interactions typically include drug receptors, drug uptake, drug breakdown, and targeted drug development. Some drugs need to attach to proteins on the surface of cells called receptors to work properly. Individual DNA determines how many and what type of receptors a person has, which can affect individual responses to the drug. This has an impact on dosage.

Some drugs need to be actively taken into the tissues and cells in which they act. Individual DNA can affect uptake of certain drugs. Decreased uptake can mean that the drug does not work as well and can cause it to problematically build up in other parts of the body. Individual DNA can also affect how quickly some drugs are removed from the cells in which they act. If drugs are removed from the cell too quickly, the drugs might not have time to fully act or to act effectively.

Individual DNA can affect how quickly a body breaks down a drug. If an individual metabolizes the drug down more quickly than a typical average person, that individual metabolizes the drug faster and may require a larger dosage of the drug or a completely different drug. Conversely, if an individual breaks the drug down more slowly, they may require a lesser dosage.

Pharmacogenomic approaches to drug development are designed to target the underlying problem rather than just treating symptoms. Some diseases are caused by specific changes or mutations in a gene. The same gene can have different types of mutations, which have different effects. Some mutations may result in a protein that does not work correctly, while others may mean that the protein is not made at all. Drugs can be created and tailored based on how the mutation affects the protein, and these drugs will only work for a specific type of mutation.

The genetic testing for cancer risk that is typically ordered by a doctor involves testing for inherited genetic variants that are associated with a high to moderate increased risk of cancer and are responsible for inherited cancer susceptibility syndromes. By contrast, direct to customer (DTC) genetic testing for cancer risk often involves the analysis of common inherited genetic variants that, individually, are generally associated with only a minor increase in risk. Even when added together, all the known common variants associated with a particular cancer type account for only a small portion of a person's risk of that cancer.

Genetic tests based on these common variants have not yet been found to help patients and their care providers make health care decisions and, therefore, they are not a part of recommended clinical practice. Whole-genome sequencing (WGS) is a comprehensive technique for analyzing entire genomes. Genomic information has been instrumental in identifying inherited disorders, characterizing the mutations that drive cancer progression, and tracking disease outbreaks.

As illustrated in FIG. 1, the system 100 comprises a server 110. A memory 130 storing a set of computer readable software instructions configured as a customer portal 140 is stored on the server 110. The consumer portal 140 allows consumers to create an account and order genetic testing. The customer portal 140 is accessible via a user access device 170 over a network 160. The user access device 170 may be a laptop computer 172, a smart phone 174, a desktop computer 176, a smart tablet, an interactive display, or any similar user input/access device. When executed by a processor 120, the server 110 accesses a request from a processor of the user access device 170 to create an individualized life assurance product for that user. The server 110 is further configured to upload a medical and behavioral history of a user to the customer portal 140.

Next, a plurality of genetic and genomic tests is suggested for the user based on an analysis of the user's unique medical and behavioral history. The plurality of genetic and genomic tests comprise at least one heritable cancer test and a pharmacogenomics test. The plurality of genetic and genomic tests may further comprise a microbiome test, an epigenetic test, and a proteome test.

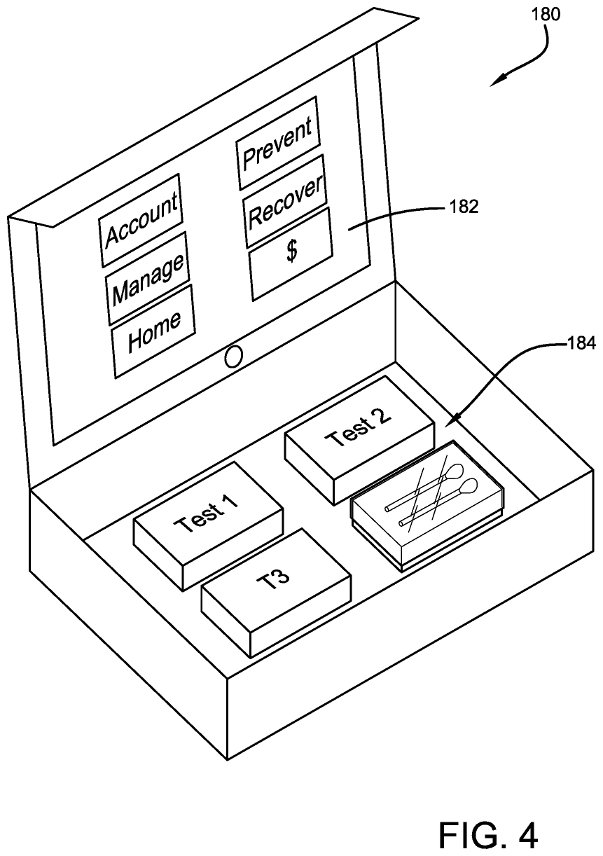

As illustrated in FIG. 4, the system 100 further comprises an at home cancer test kit 180 comprising a plurality of genetic and genomic test materials 184. A user input 182 of the test kit is used to access the server, via a test kit processor, to guide the user through a genetic and genomic sampling procedure using the plurality of genetic and genomic test materials 184. Once the samples are collected, the plurality of genetic and genomic test materials 184 are sent to a laboratory for analysis. The omics based tests help policyholders proactively manage their health and risk of cancer. The user input 182 may be an integrated video screen or smart display configured to help guide policyholders through the process of providing a DNA sample using a genetic test kit or provide further access to the consumer portal 140.

Once testing is complete, a plurality of test results are uploaded to the server 110 for analysis by the set of computer readable software instructions 140 and a user's cancer risk stratification based on the medical history, the behavioral history, and the plurality of genetic and genomic test results is generated. The test results are accessible to the user via accessing the customer portal 140. The set of computer readable software instructions of the consumer portal 140 then creates the individualized life assurance product for managing a rick of cancer based on the risk stratification analysis.

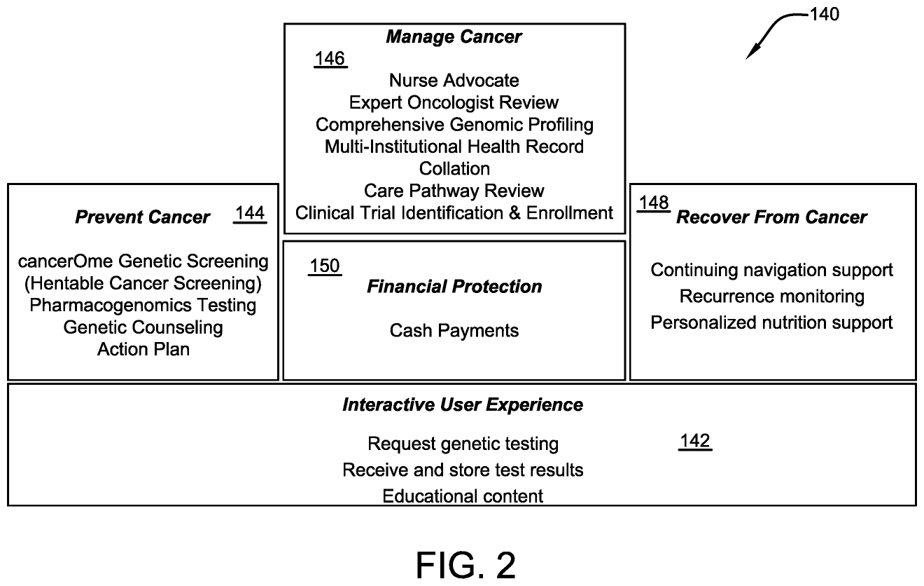

As illustrated in FIG. 2, the individualized life assurance product hosted on the customer portal 140 comprises a cancer prevention component 144, a cancer management component 146, a cancer recovery component 148, and a financial protection component 150. The components 144-150 are accessible via an interactive user experience 142 on the customer portal 140. The individualized life assurance product is adjustable based on a plurality of the user's omics markers to regularly measure a health status of the user over the life of the individualized life assurance product or by utilizing a plurality of gamifying omics-based health management tools.

The cancer prevention component 144 is configured to provide omics based testing, genetic counseling, and action plans. The cancer management component 146 is configured to provide a range of support services delivered by a precision navigator, second opinions, omics based diagnostic testing, clinical trial enrollment, nutrition support, and coaching. The cancer recovery component 148 is configured to provide residual disease monitoring and rehab services. The financial protection component 150 is configured to provide cash payouts. The precision navigator is assigned to help the policyholder navigate the health system and to organize deployment of the different services.

The precision navigator is configured to identify, as facilitated through its proprietary clinical decision tree, relevant diagnostics and existing treatment options that may be suitable for a specific policyholder following that policyholder's enrollment in the policy. Following an evaluation of documentation, data, metrics, medical and other information provided by the policyholder (the “Policyholder Metrics”), the precision navigator will engage in certain activities based on the specific policyholder's diagnosis and needs, to develop a findings report summarizing relevant diagnostics and existing treatment options suitable for policyholder. Such activities undertaken in furtherance of developing a report may include, but are not limited to, research, expert engagement, molecular tumor profiling, and clinical trial research. The report shall be shared with the policyholder, the policyholder's Medical Team when available and any other authorized individual, as designated by policyholder.

Table 1 illustrates an exemplary list of deliverables and services that may be provided via the precision navigator.

TABLE 1

| Stage | Process | Features |
|---|---|---|
| General | Navigator/Patient Communication | Navigator available for ad hoc Q&A |
| | | with patient via scheduling software |
| | | within patient portal, progress |
| | | updates and support provided via |
| | | patient preferred communication |
| | | throughout each phase of the |
| | | deliverable |
| | Patient Onboarding & Consenting | |
| Intake & Program | a) Patient portal setup to complete | Patient access to secure portal login |
| Initiation | onboarding and consenting | with consenting process to enable |
| | | navigator/patient communication |
| | | scheduling |
| | b) Introductory communication | Explanation of process/features - ~30 |
| | | minutes with navigator |
| | c) Medical record | Collection of personal and clinical |
| | intake/consolidation & review | information |
| | d) Case summary | At-a-Glance Summary of Patient Case |
| | | to-date |
| | e) GAP Analysis | Assessment of diagnosis/staging, |
| | | treatment plan, records & molecular |
| | | data, biospecimen availability |
| | f) Action Plan Communication | Patient education of efforts |
| | | pertaining to next steps, timelines, |
| | | and deliverables |
| | g) Molecular Profiling Facilitation | Requisition of molecular profiling |
| | | test(s) |
| | Program Deployment | |
| Execute | Molecular testing & analytics | Molecular results - summary of |
| | Note: The frequency and type of | actionable alterations and |
| | testing will be determined via | implications - reviewed with |
| | proprietary processes and within | patient/navigator. Tests executed |
| | the scope of the budget/timeframe | may be one or more of the following: |
| | allocated for each carrier program. | a) Germline sequencing |
| | Vendor selection will be based on | b) Somatic sequencing |
| | specimen availability/quality and | c) Therapeutic Predictive Testing |
| | urgency. | d) MRD Testing Liquid Biopsy |
| | | e) Predictive Analytics |
| | b) Second expert medical opinion | Second medical opinion - may consist |
| | | of medical, surgical, radiation, and/or |
| | | pathology |
| | c) Targeted clinical trial survey | 3-5 clinical trials with medical |
| | | record, molecular profile, & |
| | | inclusion/exclusion criteria |
| | | consideration - reviewed with |
| | | patient/navigator |
| | Reporting & Review | |
| Deliver | a) Compile “Precision Report” | Reporting (living document with |
| | | version control) |
| | | 1) 1-3 Page Report with Summary of |
| | | Clinically Relevant Information: |
| | | Actionable Alterations & |
| | | Implications, Targeted Therapy |
| | | Option(s), Second Opinion, Clinical |
| | | Trials, Case Summary |
| | | 2) Dynamic Appendix: Patient- |
| | | friendly, Glossary of Terms, |
| | | Resources for additional |
| | | information/support outside the |
| | | scope of PHN's offerings |
| | b) Navigator to patient consult | 30 min patient/navigator “precision |
| | | report” review and explanation |
| | Protection Program - Recurrence | |
| | Monitoring | |
| Monitor* | a) Recurrence Monitoring Liquid | Logistics organized; testing cadence |
| | Biopsy | maintained at recommended |
| | | frequency per patient basis for |
| | | duration of carrier program |
| | | Navigator updates and liaises with |
| | | patient to execute and report results |
| | | on each test |

Additional services may include: onboarding and consenting upon receipt of the notification of evidence of disease within oncology; unlimited access to the platform to facilitate the exchange of confidential medical information and data related to policyholder's diagnosis; access to molecular profiling to identify molecular drivers to help inform care pathway identification; access to a second medical opinion informed by available medical records and genomic information; access to clinical trial identification, aiming to identify 3-5 clinical trials relevant to the policyholder within the scope of the policyholder's trial preference; access to supplemental resources, to include but not limited to, nutrition, symptom, and wellness guidelines to improve quality of life when appropriate for the policyholder; access to a designated oncology nurse navigator and related team members to ensure a high-quality experience for the policyholder; evaluation of policyholder metrics in furtherance of the report creation; development and production of the report; and a recommendation for potential ancillary services.

The system 100 provides access to genetic counselors to chat with policyholders who wish to discuss their results. The system 100 further provides educational content such as videos and info sheets to help policyholders improve their “genetic literacy', which in turn increases awareness and engagement with the program. If a policyholder is diagnosed with cancer the system 100 provides a wide array of services designed to help increase the likelihood of a good outcome/improved mortality. The system 100 is configured with the ability to build vertical ecosystems within a specific therapeutic area and then integrate them through efficient process design and technology creating end-to-end experiences that dramatically reduce the friction of health navigation.

On a regular basis, such as triennially, the test results may be updated to ensure policyholders are risk stratified appropriately. Risk stratification is a means to help policyholders proactively manage their risk and modify their lifestyle accordingly. In addition to regular risk stratification, the system 100 also provides access to other omics based tests at a discounted rate. These tests include microbiome testing, epigenetic testing, proteome testing etc. Some tests will be made available at no cost at certain anniversary times during the life of the policy.

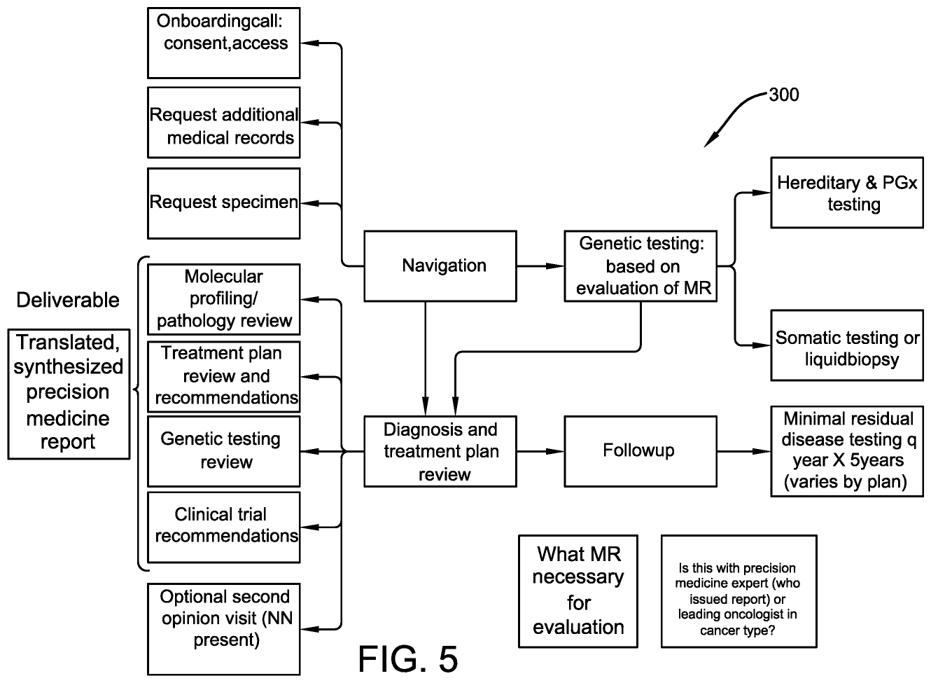

FIGS. 5-11 illustrate a method 300 for creating and administering a life assurance product for managing a risk of cancer. The method 300 employs a computer-based system 100 comprising a non-transitory set of computer readable media storing a set of computer executable instructions accessible via a user access device 170 connected to the computer-based system 100 over a network 160 to accomplish the method 300. The method 300 comprises constructing an individualized life assurance product based on a user's biological data. The use's biological data includes their genome and genetic profile.

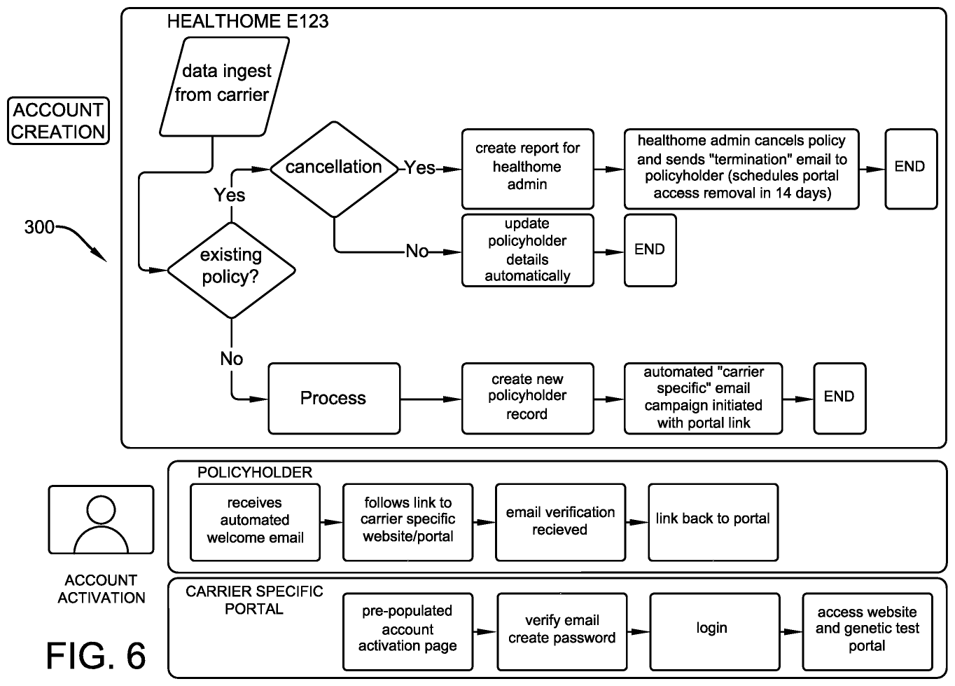

The method 300 begins by creating a customer portal 140. The customer portal 140 is created on the computer-based system 100 and is accessible via the user access device 170, via a processor 120 of the computer-based system 100. The user access device 170 may be a laptop computer 172, a smart phone 174, a desktop computer 176, a smart tablet, an interactive display, or any similar user input/access device. As illustrated in FIG. 6, the user then uses the user access device 170 to request, by a processor of the user access device 170, to create an account and the individualized life assurance product hosted on the customer portal 140. Next, the user access device 170 is used to provide and upload a medical history of the user or to provide access to the user's electronic medical records to the customer portal. Similarly, the user access device 170 is used to provide and upload a behavioral history of the user. The behavioral history may include a social or family history as well. The individualized life assurance product may be a new policy or an addition to an existing policy as discussed supra.

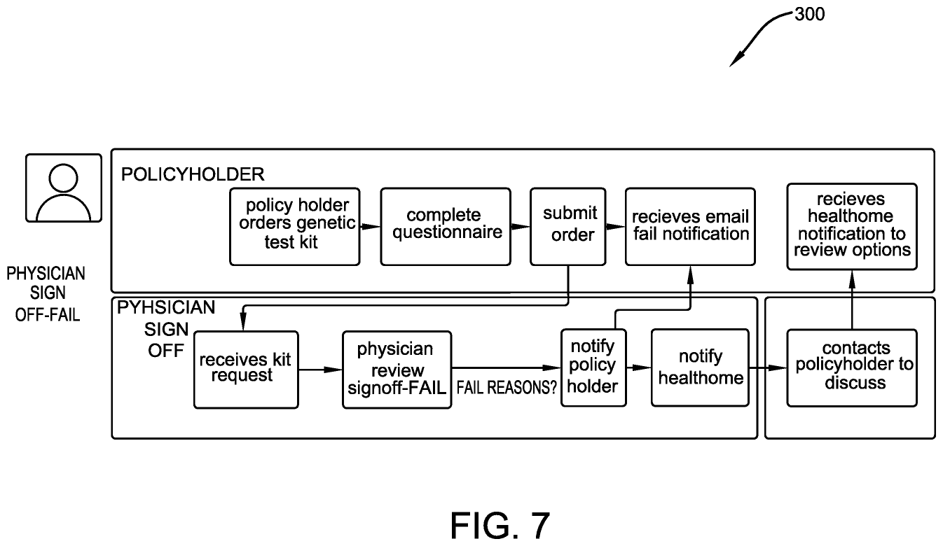

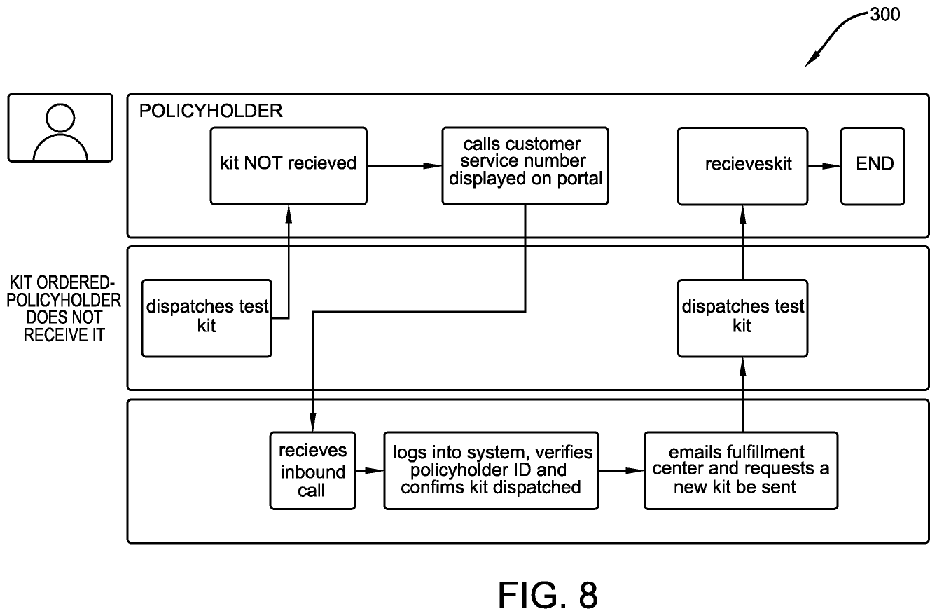

As illustrated in FIG. 7, the method 300 continues by recommending a plurality of genetic and genomic tests based on the user's biological data, medical history, and behavioral history. A medical professional may authorize or reject the testing. As illustrated in FIG. 8, a test kit 180 is ordered and sent to the user to collect biological samples that are sent to a laboratory. Once the testing is completed, a plurality of genetic and genomic test results are received from the laboratory and uploaded to the customer portal.

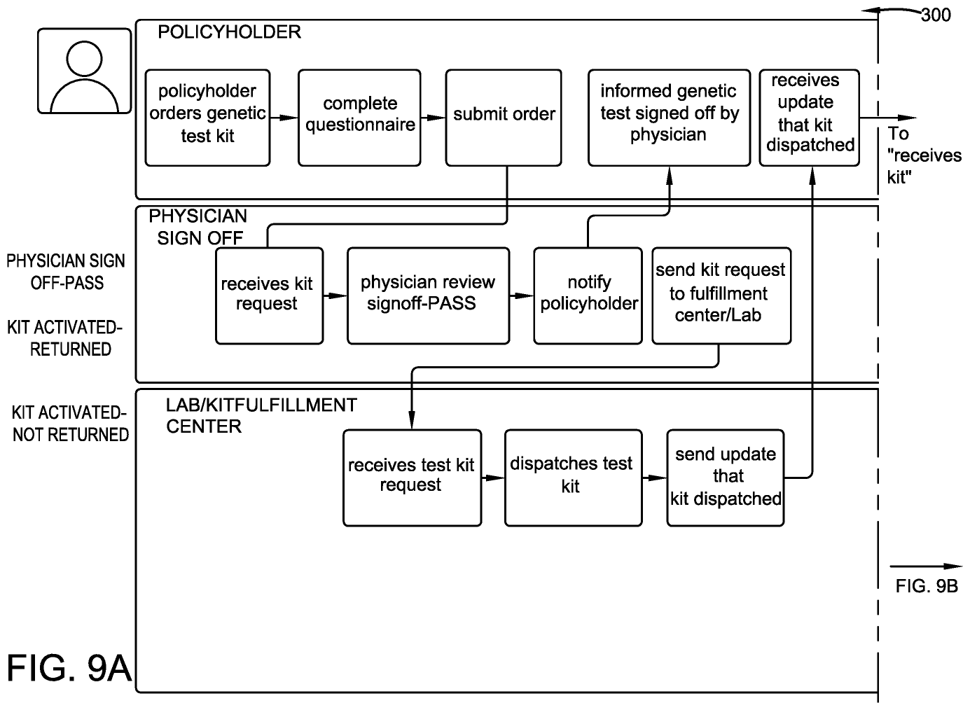

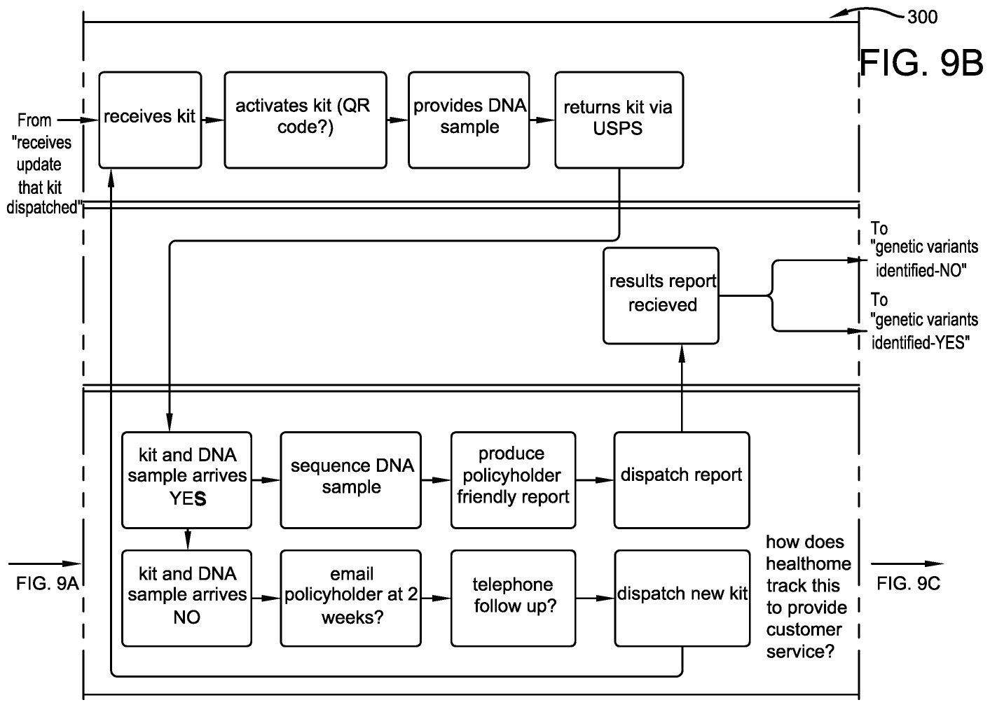

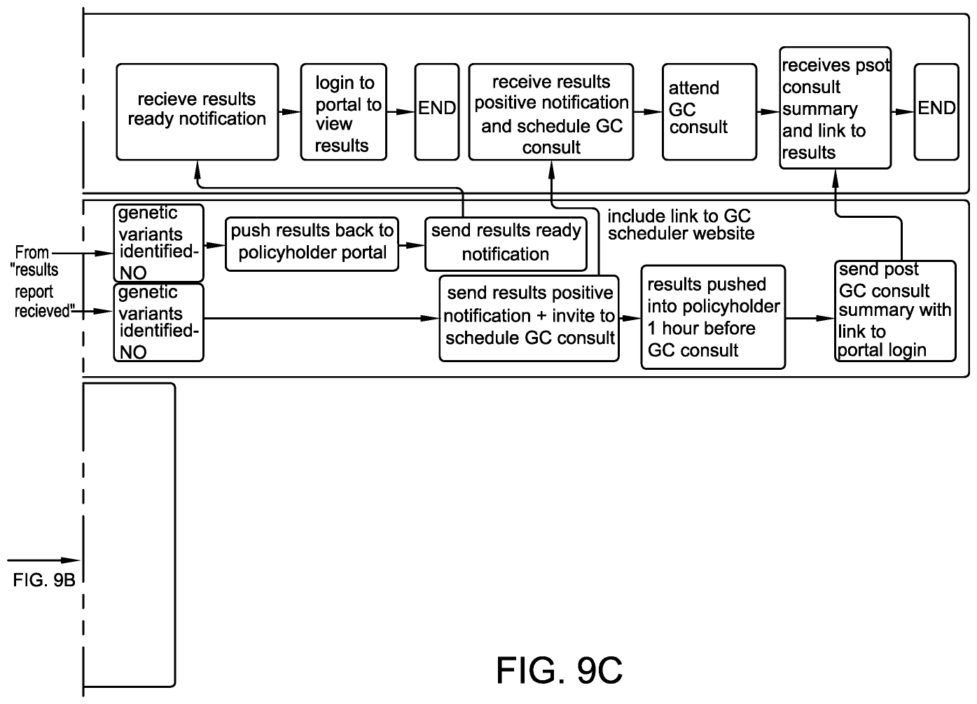

As illustrated in FIGS. 9A-9C, once the medical history, the behavioral history, and the test results are obtained, the computer-based system processes and analyzes the data and generates a risk stratification individualized to the user based on the medical history, the behavioral history, and the test results. A medical professional is provided to review, analyze, and approve the results. Next, the individualized life assurance product is created and curated based on the generated risk stratification. The individualized life assurance product may be adjusted or modified over a life of the individualized life assurance product. Adjustment may be based on additional genetic and genomic test results, a plurality of omics-based health management tools, compliance with a customized health management strategy generated by the computer-based system based on financial incentives, or by utilizing a plurality of gamifying omics-based health management tools.

The customer portal 140 comprises a prevention component 144, a disease management component 146, a disease recovery component 148, and a risk based financial protection component 150. The prevention component 144 is configured to recommend a genetic and genomic screening test plan, a genetic counseling plan, and an action plan based on the medical history, the behavioral history, and the plurality of genetic and genomic test results. The prevention component 144 is customized to the user based on the medical history, the behavioral history, and the plurality of genetic and genomic test results.

As illustrated in FIG. 5, the disease management component 146 is configured to provide a medical advocate, an expert medical review of test results, a comprehensive genetic profile, a health record collation, and a care pathway review. The disease management component 146 is customized to the user based on the medical history, the behavioral history, and the plurality of genetic and genomic test results. Disease management may include, but is not limited to, further testing, medical consulting, clinical trial recommendation, treatment plans, diagnostic and treatment plan and review, and further evaluations.

The disease recovery component 148 is configured to provide a continuing navigational support, a recurrence monitoring plan, and a personalized nutritional support. The disease recovery component is customized to the user based on the medical history, the behavioral history, and the plurality of genetic and genomic test results. The risk based financial protection component 150 provides the user a financial incentive based on the risk stratification for the user. The risk based financial protection component 150 is calculated, by the processor of the computer-based system 100, based on the risk stratification for the user based on the medical history, the behavioral history, the plurality of genetic and genomic test results, and any further input from the system 100.

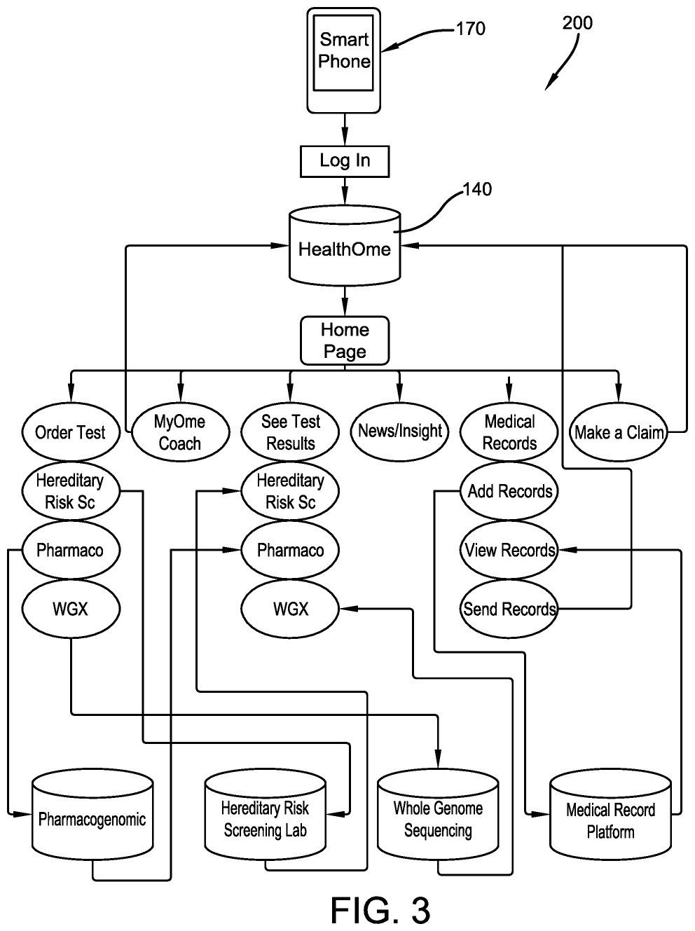

Example 1 illustrates an exemplary method 200 of using the system 100 as illustrated in FIG. 3.

### EXAMPLE 1

### Customer Journey

After purchase, a member will receive an email or communication on the first day of their coverage. The email or communication will provide the member with information regarding:
a. Confirmation of Coverage
b. Explanation of Benefits
c. Log-In to Portal
d. Next Steps—the member's “MyOme Coach” will send a separate email asking the member to schedule their orientation call. The “MyOme Coach” is a personalized licensed health care professional. (The member cannot order their test(s) until they complete their orientation call with their “MyOme Coach”). Aspects of the MyOme Coach can be automated either as a standalone application or as a tool to be used by the health care professional. The automated Coach will utilize various data driven techniques, including artificial intelligence, and machine learning technologies.

A second email will shortly be sent to the member from their assigned “MyOme Coach” with the following:
a. Personal Introduction
b. Explanation of 1-hour orientation
c. Instructions to schedule the 1-hour orientation with the “MyOme Coach” in the portal

### 1-Hr Orientation

The assigned “MyOme Coach” will call the member at the designated time for the 1-hour orientation. The “MyOme Coach” will explain the program and answer any questions the member may have. The “MyOme Coach” will begin to set up the Medical Record Platform (MRP) for the member along with asking basic medical questions to being populating the MRP.

The MyOme Coach would use an algorithm and artificial intelligence (AI) to collect Member data and tailor the MRP to the Member's profile.

The member may request their “MyOme Coach” to obtain their medical records with HIPAA compliance. The medical record consolidation process is complicated and involves complex software to do it. Lastly, the member will learn about the available DNA test available in the program and how/when to utilize each test.

The Member will receive a recap email with instructions to order their available test(s).

The system supports a Member interactive capability in order to further tune their MRP. Technically, this capability would be deployed on the system's back end for the user to manually tune their MRP.

### Test Ordering

The Member may order their test kit(s) at their convenience but is suggested to ASAP. Members may follow the link(s) in the email they received from their “MyOme Coach”. The links bring members to a secure website to answer medical questions—this needs to be completed in order for an online physical to approval test requisition. Once approved within minutes, the member may enter their mailing address where the kit will be sent.

The testing kits available to the members may include:
a. Hereditary Risk Screening Kit
b. Pharmacogenomic Testing
c. Blood marker tests, Microbiome, Proteome, etc.

### Test Result Review and Consultation:

The Member will receive an email notifying them of the completed results and a link to schedule a 45 minute meeting with their “MyOme Coach” and an Ome Genetic Counselor to review the results. The “MyOme Coach” facilitates the call between Member and Genetic Counselor and is updating MRP with notes on behalf of the Member. (This process happens for each test result, I.E. Hereditary Screening, Pharmacogenomics, and others). In use, the automated MRP engages various databases using AI to tie test results to latest diagnostic/lifestyle related information.

Based on the results, the member may ask questions to the Genetic Counselor and “MyOme Coach” or share the results with health team, if necessary. A member's first-degree family member (>18 year old) may qualify to receive the same screening, at no cost, if the Member is at high risk for certain types of cancers based on the Hereditary Risk Screening. This is only for Hereditary Risk Screening.

### Continuous Engagement <1 Year

Members may request ongoing support with their “MyOme Coach” by scheduling time allotted meetings in their portal (app 2.0). (Need time restrictions based on time period, I.E., only 1 hour per quarter). Members may use this support to ask additional questions, find specific information, guide them in the right direction of care, etc. MRP AI function will assume calendaring function. Can also create auto appointment feature tying in with other systems/databases to manage patient appointments/follow ups.

Members will also receive personalized emails from their MyOme Coach with the following:
a. Check-in to remind Member they can schedule time to ask questions
b. Send new and relative information about Omics and other sciences in medicine
c. Send members health tips
d. Send updates on new available testing technology available to the m

### >1 Year Engagement

Members will receive the same services as they received in year one, minus retaking the Pharmacogenomic and Hereditary Risk Screening Kit. However, they will be offered a Whole Exome Test on the anniversary of their second year of the program. Once the Whole Exome Test is complete, the member may upload their VCF file to new genomic data bases to learn new information (fitness, diet, other diseases, etc). The Member may upload their VCF up to two different reports over the course of the year at no additional cost. (Ome may sell a whole exome test if a member wants one prior to the first year anniversary, however, the benefit of waiting in the program is the cost of the Whole Exome Test is covered in the program along with additional services).

### Diagnosis

Members should contact their MyOme Coach at their earliest convenience to report a diagnosis of cancer or other covered illness. Their MyOme Coach will explain the claim's process to the member and will explain the support services available to the member. (Claim process—paperwork that needs to be completed by member and their health team)

The services available to members upon a diagnosis include:
a. Assistance with second opinion at no cost
b. Initial cash payout
c. Assistance with second pathology opinion at no cost
d. Nurse navigator—we send with a member to their first appointment to help explain medical jargon
e. Comprehensive Genomic Profiling
f. Whole Genome Sequencing
g. Members may significantly increase access to important and potentially lifesaving clinical trial via Clinical Trial Navigator
h. Protocol checking using AI that checks diagnosis against recommended treatment protocols.
i. AI function ties in diagnosis to those hospitals/doctors that are considered leading researchers/clinicians in the area of oncology based on patient locale and national/international status.
j. AI function ties Member into information resources, such as patient list serves, tied to the diagnosed type of cancer.
k. Member receives second check of $5,000 on 6-month anniversary with continued engagement.
k. Final cash payment at 1 year anniversary occurs at the earlier of cure or death.

### Data

Member information will be gathered in order to provide a full picture of Member health experiences. That data will include Data that will follow the Member across their lifespan, and irrespective of the various institutions, hospitals and doctors that they may use. Subject to HIPAA and the Member consent, the Data can include a Member's medical data, pharmacogenomic data and their genomic test information as well as other test results that the Member may receive over time. Aggregate Member data can also be sorted on an anonymized basis. Data will be reliant on third party clinician or lab data.

### Recovery

The Member is eligible to stay in the program for the same monthly prices and will receive the ongoing support and technology of the program. They may also be paid out for a second diagnosis of the same illness or other. Members who continue in the program will no longer receive one time test such as Hereditary Risk Screening, Pharmacogenomics, Whole Exome Sequencing, and others. However, Members who continue the program may receive additional Germ Line testing (Comprehensive profiling test) as needed for their care at no additional cost.

The program will also monitor and MyOme Coach will assist in coordinating cancer survivor issues, e.g. survivor testing, appointment coordination, provision of survivor studies and programs information.

It is also contemplated that the system 100 is configured with a focus on cardiovascular health with genetics and epigenetics testing that assesses the near-term (approximately 3-year) risk for a heart attack. The system 100 may also include a range of support services to help policyholders reduce their risk of cardiac heart disease with services designed to support a policyholder after they have had a cardiovascular event such as a heart attack, stroke etc. The epigenetic test may be re-deployed every three years. The system 100 is designed to form a policy that will become known as a life “assurance” policy. The concept of life assurance is to create a “life and health optimization” experience that helps policyholders live longer and healthier lives. The proprietary software of the system 100 may gamify managing health using omics markers to measure health status.

Various implementations of the systems and techniques described here can be realized in digital electronic circuitry, integrated circuitry, computer hardware, firmware, are described as having, including, or comprising specific software, and/or combinations thereof. These various implementations can include implementation in one or more computer programs that are executable and/or interpretable on a programmable system including at least one programmable processor, couples to receive data and instructions from, and to transmit data and instructions to a storage system, at least one input device, and at least one output device.

These computer programs (also known as programs, software, software applications or code) include machine instructions for a programmable processor and can be implemented in a machine or programming language. As used herein, the terms machine-readable medium and computer-readable medium refer to any computer program product, apparatus and/or device used to provide machine instructions and/or data to a programmable processor, including a machine-readable medium that receives machine instructions as a machine-readable signal. The term machine-readable signal refers to any signal used to provide machine instructions and/or data to a programmable processor.

It is contemplated that the system 100 and methods 200, and 300 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

Notwithstanding the forgoing, the system 100 and methods 200, and 300 of the present invention and its various structural components can be of any suitable size, shape, and configuration as is known in the art without affecting the overall concept of the invention, provided that it accomplishes the above stated objectives. One of ordinary skill in the art will appreciate that the shape and size of the system 100 and methods 200, and 300 and its various components and material, as shown in the FIGS. are for illustrative purposes only, and that many other shapes and sizes of the system 100 and methods 200, and 300 are well within the scope of the present disclosure. Although the dimensions of the are important design parameters, the system 100 and methods 200, and 300 and its components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method creating and administering a life assurance product on a computer-based system accessible via a user access device connected to the computer-based system over a network, the method comprising:
constructing an individualized life assurance product based on a user's biological data by:
creating a customer portal on the computer-based system accessible via the user access device;
receiving, by a processor of the computer-based system, from a processor of the user access device, a request from the user access device to create the individualized life assurance product;
providing, by the processor of the user access device, a medical history of the user to the customer portal;
providing, by the processor of the user access device, a behavioral history of the user to the customer portal;
obtaining a plurality of genetic and genomic test results based on the user's biological data;
generating, by the processor of the computer-based system, a risk stratification for the user based on the medical history, the behavioral history, and the plurality of genetic and genomic test results;
curating the individualized life assurance product based on the generated risk stratification; and
adjusting the individualized life assurance product over a life of the individualized life assurance product based on additional genetic and genomic test results and a plurality of omics-based health management tools.

2. The method of claim 1, wherein the life assurance product is a cancer-based life assurance product.

3. The method of claim 1, wherein the life assurance product comprises a critical illness policy, a life insurance policy, or a hybrid critical illness and life insurance policy.

4. The method of claim 1, wherein the plurality of genetic and genomic test results comprise at least one heritable cancer test and a pharmacogenomics test.

5. The method of claim 1, wherein the plurality of genetic and genomic test results comprise a microbiome test, an epigenetic test, and a proteome test.

6. The method of claim 1 further comprising adjusting the individualized life assurance product over the life of the individualized life assurance product based on compliance with a customized health management strategy.

7. The method of claim 1, wherein the customer portal comprises a prevention component customized to the user based on the medical history, the behavioral history, and the plurality of genetic and genomic test results.

8. The method of claim 7, wherein the prevention component is configured to recommend a genetic and genomic screening test plan, a genetic counseling plan, and an action plan based on the medical history, the behavioral history, and the plurality of genetic and genomic test results.

9. The method of claim 1, wherein the customer portal comprises a disease management component customized to the user based on the medical history, the behavioral history, and the plurality of genetic and genomic test results.

10. The method of claim 9, wherein the disease management component is configured to provide a medical advocate, an expert medical review of test results, a comprehensive genetic profile, a health record collation, and a care pathway review.

11. The method of claim 1, wherein the customer portal comprises a disease recovery component customized to the user based on the medical history, the behavioral history, and the plurality of genetic and genomic test results.

12. The method of claim 11, wherein the disease recovery component is configured to provide a continuing navigational support, a recurrence monitoring plan, and a personalized nutritional support.

13. The method of claim 1 further comprising calculating, by the processor of the computer-based system, a risk based financial protection component based on the risk stratification for the user based on the medical history, the behavioral history, and the plurality of genetic and genomic test results.

14. The method of claim 1 further comprising adjusting the individualized life assurance product utilizing a plurality of gamifying omics-based health management tools.

15. A system for creating and administering a life assurance product for managing a risk of cancer, the system comprising:
a server comprising a memory storing a set of computer readable software instructions configured as a customer portal, that when executed by a processor, causes the server to:
access a request from a processor of a user access device to create an individualized life assurance product;
upload a medical and behavioral history of a user to the customer portal;
suggest plurality of genetic and genomic tests for the user based on the user's medical and behavioral history;
generate a user's cancer risk stratification based on the medical history, the behavioral history, and the plurality of genetic and genomic test results;
create an individualized life assurance product for managing a rick of cancer based on the risk stratification; and
wherein the individualized life assurance product comprises a cancer prevention component, a cancer management component, a cancer recovery component, and a financial protection component accessible via the customer portal.

16. The system of claim 15, wherein the system further comprises an at home cancer test kit.

17. The system of claim 16, wherein the at home cancer test kit comprises a user input, that when executed by a test kit processor, accesses the server.

18. The system of claim 16, wherein the at home cancer test kit comprises a plurality of genetic and genomic test materials.

19. The system of claim 14, wherein the individualized life assurance product is adjustable based on a plurality of the user's omics markers to regularly measure a health status of the user over the life of the individualized life assurance product.

20. A non-transitory set of computer readable media storing a set of computer executable instructions that, when executed by a computing device, causes the computing device to:
receive a medical and behavioral history of a user to the computing device from a user input device;
suggest plurality of genetic and genomic tests for the user based on the user's medical and behavioral history;
analyze the medical and behavioral history and the genetic and genomic test results and generate a user's cancer risk stratification;
create an individualized life assurance product for managing a risk of cancer based on the risk stratification;
modify the individualized life assurance product based on compliance with a plurality of recommended actions based on the user's cancer risk stratification; and
provide a financial incentive to comply with a plurality of recommended actions.
